(19) United States
(12) Patent Application Publication
(10) Pub. No.: US 2023/0263965 A1
(43) Pub. Date: Aug. 24, 2023

(54) PORTABLE NEBULIZER

(71) Applicant: AeProMED Innovation Co., Ltd., Zhubei City (TW)

(72) Inventors: Huang-Te LI, Zhubei City (TW); Yi-Tzu CHANG, Zhubei City (TW); Yung-Ying CHEN, Zhubei City (TW); I-Hui WU, Zhubei City (TW)

(21) Appl. No.: 17/678,252

(22) Filed: Feb. 23, 2022

(51) Int. Cl. A61M 11/00; A61M 35/00

(52) U.S. Cl. CPC A61M 11/006; A61M 35/003

(57) ABSTRACT

A portable nebulizer provided by the invention is particularly suitable for using with a liquid contained in a capsule or in a container made of a disposable material that can be easily damaged, the portable nebulizer comprises a driving unit for driving an atomizing unit, and a containing unit for containing the container, the containing unit has a breaking part for breaking the container to facilitate outflow of the liquid, after the liquid flows into an inner space of a main body of the containing unit because the container is damaged by the breaking part, the liquid can be atomized by the atomizing unit to produce a mist capable of escaping to the outside of the main body for a user to use the liquid mist conveniently.

### BACKGROUND OF THE INVENTION

### Field of Invention

The invention relates to medical/cosmetic appliances, and more particularly to a portable nebulizer capable of atomizing liquid.

### Related Art

In the prior art related to the nebulizer provided by U.S. Pat. Publication No. 20190240428, the inner cup of a specific volume can be pre-inserted into the accommodating space of the medicament container as a space for containing a specific amount of medicament, so that excessive medicament overflowed from the inner cup can be collected and separated in other spaces of the medicament container to avoid filling too much medicament and causing overdose.

Although the above-mentioned technology can ensure the correctness of dosage of medicament for the patient in a respiratory therapy of medicament atomization, it is still inconvenient in actual use, especially in the filling of medicament, insufficient dosage will affect the effect of treatment, although overdose can be prevented from being administered to the patient by the volume limitation of the inner cup, the overflowed medicament will be treated as a waste, resulting in a drawback of wasting medical resources.

In addition, the conventional nebulizers can only correspond to a single medicament, so when there is a need for two or more than two kinds of drugs, it is necessary to carry multiple nebulizers at the same time, which leads to inconvenience in use; and currently, besides medical use, other fields such as cosmetology or nursing also need to provide active ingredients to users through nebulizers, but due to the structural design of the conventional nebulizers, the conventional nebulizers cannot be used for multiple purposes at the same time.

### SUMMARY OF THE INVENTION

Therefore, a main object of the invention is to provide a portable nebulizer capable of ensuring a correct dosage of a medicinal liquid or other liquids to avoid waste, and at the same time, filling of the liquid can be carried out conveniently.

Another object of the invention is to provide a portable nebulizer capable of replacing an atomized liquid according to the requirements of use, and the liquids will not contaminate one another so as to achieve an efficacy of increasing the convenience and applicability of use.

In order to achieve the above-mentioned objects, the portable nebulizer provided by the invention is particularly suitable for using with a liquid contained in a capsule or in a container made of a disposable material that can be easily damaged, the portable nebulizer comprises a driving unit for driving an atomizing unit, and a containing unit for containing the container, the containing unit has a breaking part for breaking the container to facilitate outflow of the liquid, after the liquid flows into an inner space of a main body of the containing unit because the container is damaged by the breaking part, the liquid can be atomized by the atomizing unit to produce a mist capable of escaping to the outside of the main body for a user to use the liquid mist conveniently.

Wherein the liquid contained in the container can be a medicament, a solution containing plant extracts, a water-based solution, a beauty serum or other functional liquid mixtures.

For example, if the liquid contained in the container is a medicament, a user can use the medicament in a specific dose package according to a prescription directly with the portable nebulizer of the invention to eliminate the inconvenience of having to obtain a medicinal liquid of a medicament and then fill the medicinal liquid as required in the prior art.

In order to facilitate replacement or cleaning of the containing unit, the containing unit and the combined driving unit can be conveniently separated and combined, the driving unit has a body and a first joint part disposed on the body, and the containing unit has a main body and a second joint part disposed on the main body, so that the first joint part and the second joint part can be combined with or separated from each other to achieve combination or separation between the containing unit and the driving unit.

Wherein the first joint part and the second joint part are respectively a groove and a flange with a complementary concavo-convex configuration.

In order for the breaking part to break the container, a breaking end of the breaking part has a needle-like, sheet-like or rib-like structure with a strength sufficient to be capable of damaging an integrity of a container wall made of plastic or other disposable materials that can be easily broken through by puncturing, piercing, cutting or slashing or other similar effects to achieve an object of enabling the medicinal liquid in the container to flow out.

Further, in order to facilitate the driving unit to drive the atomizing unit, the driving unit can be provided with a first electrical connection part disposed on the body, and the containing unit can be provided with a second electrical connection part disposed on the main body, when the containing unit and the driving unit are combined with each other by the second joint part and the first joint part, the first electrical connection part and the second electrical connection part abut against each other to form an electrical connection, thereby the driving unit is capable of driving the atomizing unit.

Wherein the electrical connection between the first electrical connection part and the second electrical connection part can be used for electric current and/or signal transmission.

The atomizing unit is embedded in the main body of the containing unit, and is located in a passage of the containing unit for communicating the inner space inside the main body for accommodating the medicament with the outside, and is controlled by the main body via the second electrical connection part, so that the medicinal liquid of the medicament can be atomized by the atomizing unit through the passage, and then released externally in a mist state for a patient to use.

### BRIEF DESCRIPTION OF THE DRAWINGS

FIG. 1 is a perspective view of a preferred embodiment of the invention.

FIG. 2 is an exploded view of a preferred embodiment of the invention.

FIG. 3 is a partial perspective view of a preferred embodiment of the invention.

FIG. 4 is a partially exploded view of a preferred embodiment of the invention.

FIG. 5 is a cross-sectional view of a preferred embodiment of the invention along the section line 5-5 in FIG. 3.

FIG. 6 is a cross-sectional view of a preferred embodiment of the invention along the section line 6-6 in FIG. 5.

FIG. 7 is a perspective view of the use of a preferred embodiment of the invention.

FIG. 8 is another perspective view of the use of a preferred embodiment of the invention.

### DETAILED DESCRIPTION OF THE INVENTION

The invention provides a portable nebulizer capable of providing a user with substances of different efficacies contained in a capsule or in a container made of other easily damaged disposable materials to achieve an efficacy of treatment, health maintenance or cosmetology; in other words, the user can select a container containing a liquid according to his or her needs, and the liquid can be atomized by the portable nebulizer to contact the user or to be inhaled by the user.

For example, when the user wants to take care of or beautify his or her skin with the portable nebulizer, the user can choose a container containing a beauty serum or plant extracts, through an atomization process, the beauty serum or plant extracts will be atomized to contact the skin to achieve an efficacy of cosmetology.

Since the portable nebulizer disclosed in the invention is related to personal beauty or health, the portable nebulizer is capable of confirming the user’s identity by means of identification technology to ensure the safety and hygiene of use, and with information processing technology, the user can be provided with personalized analysis results to increase an effectiveness of treatment, health maintenance or cosmetology.

In the following, the liquid contained in the container is taken as a medicament as an example, detailed descriptions are provided hereinafter in conjunction with the accompanying drawings.

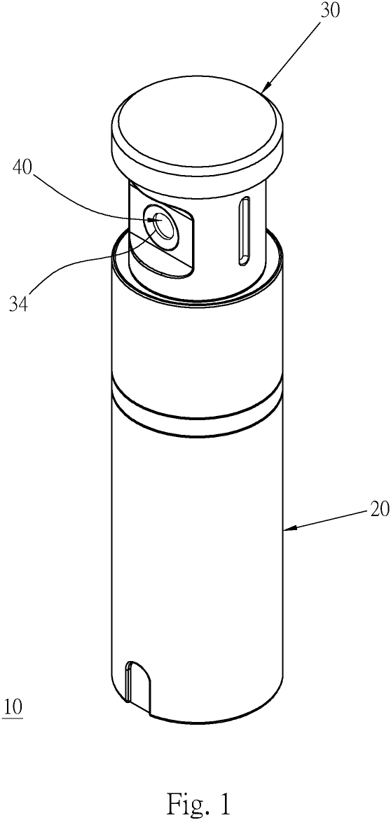

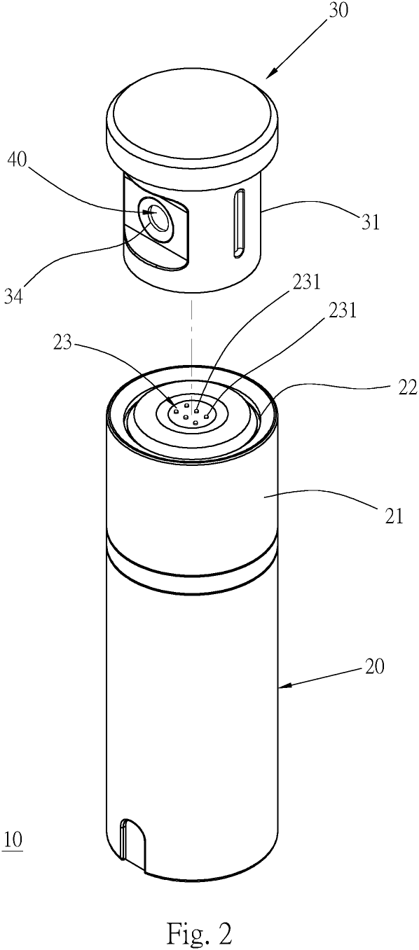

First of all, please refer to FIG. 1 and FIG. 2, a portable nebulizer 10 provided in a preferred embodiment of the invention mainly comprises a driving unit 20, a containing unit 30 and an atomizing unit 40.

As shown in FIG. 2, the driving unit 20 has a body 21 with a cylindrical outer periphery and a hollow interior for accommodating a power source and related control circuits therein; a first joint part 22 in the shape of an annular groove recessed on a top end face of a cylindrical shaft of the body 21; and a first electrical connection part 23 disposed on the top end face of the cylindrical shaft of the body 21 and located on an inner side the first joint part 22 and corresponding to a center of curvature of the body 21, wherein the first electrical connection part 23 has a plurality of electrical contact points 231 respectively electrically connected with the control circuits located inside the body 21.

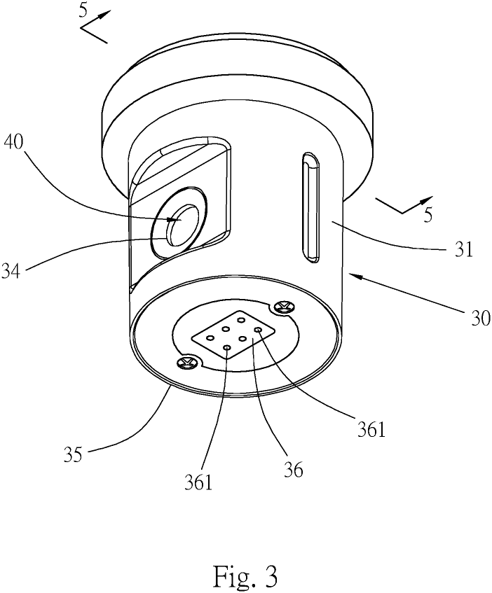

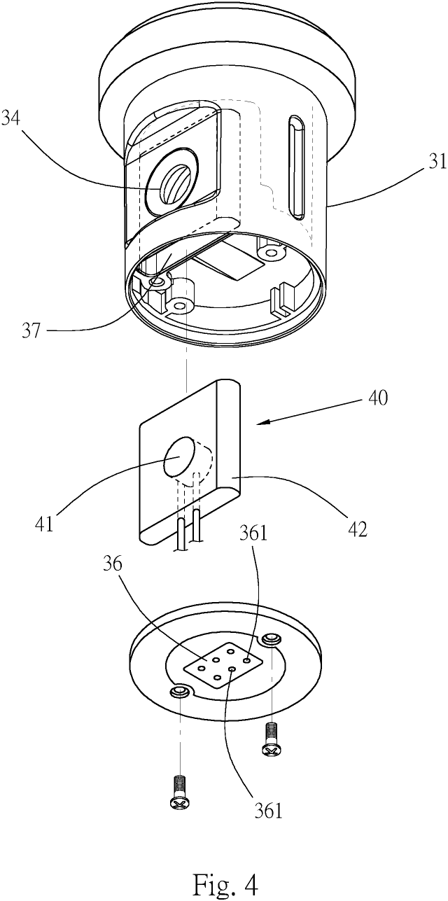

Please refer to FIG. 3, FIG. 4 and FIG. 5, the containing unit 30 has a cylindrical main body 31 with a hollow top end; an inner space 32 formed inside the cylindrical main body 31; and an opening 33 formed at a top end of the cylindrical main body 31 to communicate with the inside and the outside of the inner space 32. At the same time, the containing unit 30 further comprises a hole-shaped passage 34 penetrating through a cylindrical wall on one side of the main body 31 to communicate the inner space 32 with the outside of the main body 31; a second joint part 35 in the shape of an annular flange coaxial with the main body 31 and axially protruding from an axial bottom end of the main body 31 and with a diameter the same as that of the first joint part 22; and a second electrical connection part 36 fixedly disposed on the axial bottom end of the main body 31 and located within an annular shape of the first joint part 22.

As shown in FIG. 1 and FIG. 2, when the containing unit 30 and the driving unit 20 are coaxially embedded with each other through the concavo-convex shapes of the first joint part 22 and the second joint part 35 complementary to each other, the electrical contact points 231 of the first electrical connection part 23 are synchronously abutted against a plurality of electrical contact points 361 of the second electrical connection part 36 to form an electrical connection state.

As shown in FIG. 4, the containing unit 30 further comprises an insert groove 37 extending from a peripheral hole wall of a middle section of the passage 34 toward an interior of the main body 31, and a hollow groove opening is formed on the axial bottom end of the main body 31.

The atomizing unit 40 is provided with a conventional atomizing element 41, which is located in the insert groove 37 and corresponds to a hole axis of the passage 34, and is used for atomizing the liquid in the passage 34 flowed from an inlet direction of the passage 34 that communicates with the inner space 32, and an atomized mist is capable of escaping through an outlet end side of the passage 34 that communicates with the outside of the main body 31; and an anti-leakage and fixing layer 42 interposed between the atomizing element 41 and a groove wall of the insert groove 37 for wrapping and positioning the atomizing element 41 to prevent liquid leakage.

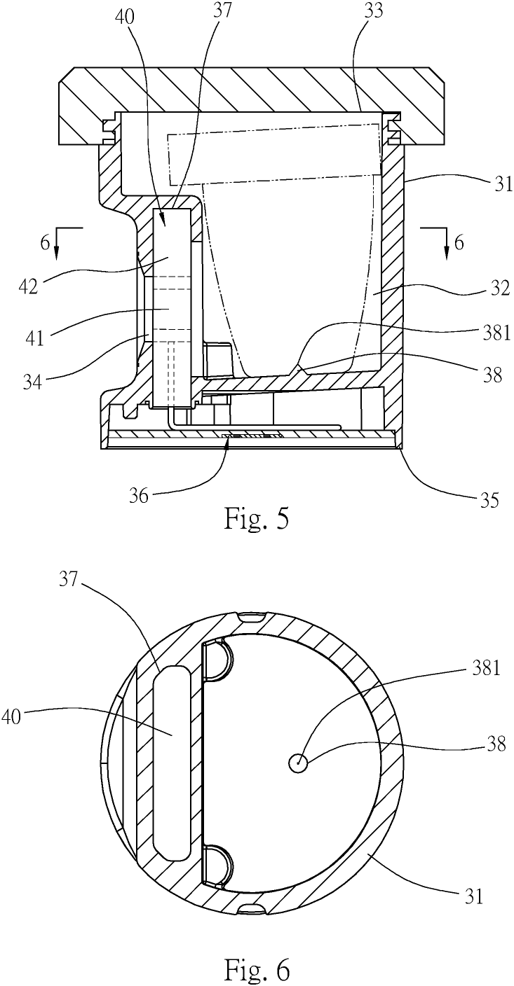

The portable nebulizer 10 is particularly suitable for medicaments that use a capsule or a container made of other easily damaged disposable materials for containing. As indicated by dotted lines shown in FIGS. 5 and 6, a medicament is directly placed in the inner space 32, under an action of an appropriate external force, the container of the medicament is damaged in the inner space 32, and the medicament liquid flows out and is contained in the inner space 32, and then the medicament liquid flows to the atomizing unit 40 through the passage 34 and escapes externally after being atomized. Thereby, a patient can directly place a specific dose of medicament into the inner space 32, without the inconvenience of measuring with a measuring cup first and then filling it from outside as required in the prior art, so that the correctness of a prescribed dose can be ensured; wherein a structure of the portable nebulizer 10 used for breaking the container of the medicament is a breaking part 38 provided on the main body 31, and the breaking part 38 is formed with a breaking end 381 protruding into the inner space 32, thereby, when the medicament is placed in the inner space 32, an additionally exerted appropriate external force is capable of causing the container of the medicament to be pierced through by the breaking end 381, so that the medicament liquid is capable of flowing out.

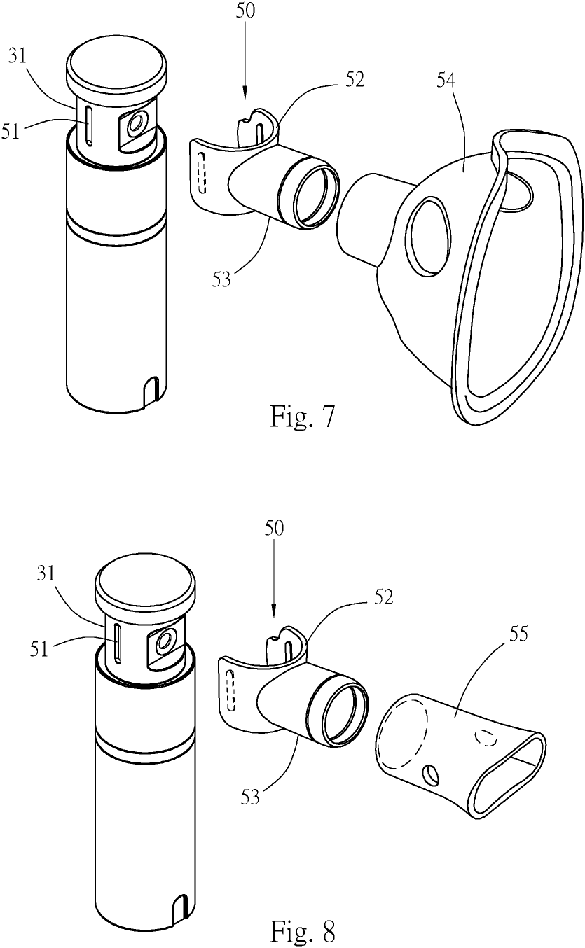

Please refer to FIG. 7 and FIG. 8, in order to facilitate the patient to inhale the atomized medicinal liquid, the portable nebulizer 10 further comprises a guide unit 50 for collecting and guiding the medicinal liquid released in the form of mist through the passage 34 to be provided for use by the patient, wherein the guide unit 50 has a pair of third connecting parts 51 being parallel concave strips disposed on two sides of the main body 31 and parallel to the hole axis of the passage 34; a fourth connecting part 52 in the shape of an arcuate sheet with two arcuate ends embedded into each of the third connecting parts 51 respectively and with a concave surface attached on the main body 31; and an extension tube 53 with one end fixedly disposed on the fourth connecting part 52 and coaxial with the passage 34, and communication between a tube opening of one end of the extension tube 53 and the passage 34 is not blocked by the fourth connecting part 52; thereby the medicinal liquid atomized by the atomizing unit 40 can be collected by the extension tube 53, and according to the needs of the user, a breathing mask 54 as shown in FIG. 7 or a breathing nozzle 55 as shown in FIG. 8 can be sleeved on another end of a tube axis of the extension tube 53 for the convenience of use by the patient.

Among the technical features of the present invention described in the above-mentioned embodiments, it should be specifically pointed out that a shape of the breaking part 38 or a way of the breaking part 38 breaking the medicament container is not limited to the above, the breaking part 38 can also be sheet-like or rib-like or in other forms with a structure and a strength sufficient to be capable of physically interfering with the container in a process of being placed in the inner space 33, and resulting in breaking the container to achieve the requirements of the invention, and with a fixed dose of medicament in a specific package to achieve efficacies of convenience of use and correct dosage.

In addition, the applicant needs to specifically point out that in order to ensure the correctness of use, the portable nebulizer 10 is capable of checking with medicament information to be used by fingerprints or other person identification techniques to ensure the correctness of a prescription object, and completing use records to assist a progress of a treatment course in order to achieve efficacies of medicament inquiry, medicament records and tracking, and dose control and regulation. A specific technical means used in the portable nebulizer 10 to achieve the above-mentioned efficacies can be realized by connecting the portable nebulizer 10 with an application program in a smart phone through a suitable connection technology, and the user’s fingerprint can be identified and obtained via a fingerprint identification element pre-disposed on the portable nebulizer 10, and compared with fingerprint records in a database, and the portable nebulizer 10 can be activated to perform the above-mentioned atomization only if the user’s fingerprint matches with the fingerprint records.

In addition to controlling activation and deactivation of the portable nebulizer, the above-mentioned application program installed on the smart phone can also be used as a provider of medicament information, enabling the user to know information of the medicament according to a doctor’s advice and to record usage footprints and related information of the portable nebulizer, or prescription can be directly adjusted to achieve an efficacy of integrating medicament with equipment.

In addition to what has been described above, with technical contents of the physical combination between the first joint part and the second joint part, and the electrical connection between the first electrical connection part and the second electrical connection part, the portable nebulizer is capable of providing more functions, for example, a detection unit provided with a nitric oxide detection element can replace the containing unit and can be combined on the driving unit through the above-mentioned technical means of physical connection and electrical connection, and through an appropriate identification means, the driving unit can be informed that it is being connected with the detection unit. In this way, the user can operate by using the above-mentioned application program and exhale at the detection unit to enable the detection unit to detect a nitric oxide index in an exhaled breath and write the index into a database to be used as a record of information related to the user’s personal health. Indeed, the detection unit can also be integrated into the containing unit without replacement to facilitate detection of nitric oxide before and after medication.

The above-mentioned embodiments are merely used to illustrate the technical ideas and features of the invention, with an object to enable any person having ordinary skill in the art to understand the technical content of the invention and implement it accordingly, the embodiments are not intended to limit the Claims of the invention, and all other equivalent changes and modifications completed based on the technical means disclosed in the invention should be included in the Claims covered by the invention.

What is claimed is:

1. A portable nebulizer comprising:
a driving unit having a body, a first joint part located on one side of the body, and a first electrical connection part located on one side of the body and separated from the first joint part;
a containing unit having a hollow main body, a second joint part capable of combining with or separating from the first joint part and located on one side of the main body, a second electrical connection part capable of connecting with or separating from the first electrical connection part and located on one side of the main body and separated from second joint part, an opening and a passage respectively provided in the main body and respectively communicating with an inner space of the main body and the outside of the containing unit, and a breaking part disposed on the main body and formed with a protruding breaking end on a chamber wall of the inner space of the main body; and
an atomizing unit disposed in the main body and lying across the passage and driven by the driving unit for atomizing a liquid in the passage flowed from an inlet direction of the passage communicating with the inner space of the main body, and an atomized mist being capable of escaping to the outside of the main body in an outlet direction of the passage;
thereby when a capsule or a container made of a disposable material containing the liquid being inserted into the inner space of the main body through the opening, the container being pierced through and formed with a hole by the breaking end with application of an external force, the liquid being capable of flowing out from the hole into the inner space of the main body, and then escaping externally after being atomized by the atomizing unit.

2. The portable nebulizer as claimed in claim 1, wherein the body is in the shape of a column, and the first joint part and the first electrical connection part are located on one end of a column shaft of the body.

3. The portable nebulizer as claimed in claim 1, wherein the first joint part and the second joint part are respectively an annular groove and an annular flange with concavo-convex shapes complementary to each other.

4. The portable nebulizer as claimed in claim 2, wherein the first joint part and the second joint part are respectively an annular groove and an annular flange with concavo-convex shapes complementary to each other.

5. The portable nebulizer as claimed in claim 3, wherein the first electrical connection part and the second electrical connection part respectively have a plurality of electrical contact points capable of abutting against one another.

6. The portable nebulizer as claimed in claim 4, wherein the first electrical connection part and the second electrical connection part respectively have a plurality of electrical contact points capable of abutting against one another.

7. The portable nebulizer as claimed in claim 1, further comprising a guide unit detachably combined on the main body to collect and guide the mist escaping from the main body.

8. The portable nebulizer as claimed in claim 7, wherein the guide unit has a third connecting part disposed on the main body, a sheet-shaped fourth connecting part capable of combining with or separating from the third connecting part, and an extension tube with a tube axial end fixedly disposed on the fourth connecting part, and a tube opening of one end of the extension tube is not blocked by the fourth connecting part, thereby the fourth connecting part is connected on the main body through combination between the fourth connecting part and the third connecting part, and the tube opening of one end of the extension tube is coaxial with an outlet end of the passage, so that the mist is capable of being collected in the extension tube.

9. The portable nebulizer as claimed in claim 8, wherein the guide unit further comprises a breathing mask detachably connected in series on another tube axial end of the extension tube.

10. The portable nebulizer as claimed in claim 8, wherein the guide unit further comprises a breathing nozzle detachably connected in series on another tube axial end of the extension tube.

11. The portable nebulizer as claimed in claim 1, wherein the breaking part is in the shape of a needle, a column, a sheet or a rib.

12. The portable nebulizer as claimed in claim 1, wherein the containing unit further comprises an insert groove extending from a peripheral wall surface of a middle section of the passage toward an interior of the main body to reach a predetermined depth for accommodating the atomizing unit.
